(19) United States
(12) Patent Application Publication
(10) Pub. No.: US 2022/0354894 A1
(43) Pub. Date: Nov. 10, 2022

(54) Cell Composition Comprising Radiolabled Mesenchymal Stem Cells, Use Thereof and Method for Preparing Radiolabeled Mesenchymal Stem Cells

(71) Applicant: Boehringer Ingelheim Veterinary Medicine Belgium, Evergem (BE)

(72) Inventors: Charlotte BEERTS, Evergem (BE); Jan SPAAS, Evergem (BE); Eva DEPUYDT, Evergem (BE)

(21) Appl. No.: 17/763,078

(22) PCT Filed: Sep. 25, 2020
(86) PCT No.: PCT/EP2020/076938

§ 371 Date: Mar. 23, 2022

(30) Foreign Application Priority Data
Sep. 27, 2019 (EP) 19200152.7

(51) Int. Cl. A61K 35/28; A61K 51/12; A61K 9/00

(52) U.S. Cl. CPC A61K 35/28; A61K 51/1203; A61K 9/0019

(57) ABSTRACT

The current invention relates to a cell composition comprising isolated mesenchymal stem cells, wherein said MSCs are radiolabeled with technetium-99m, wherein said radiolabel is essentially free of hexamethylpropylene amine oxime, and wherein said radiolabeled MSCs have a post-labeling cell viability of at least 55%. The invention also relates to the use of said cell composition for biodistribution, diagnostics and cell-based therapies. Furthermore, the invention relates to a method for preparing radiolabeled mesenchymal stem cells.

### FIELD OF THE INVENTION

The present invention relates to a cell composition comprising isolated mesenchymal stem cells, wherein said MSCs are radiolabeled with technetium-99m. The invention further relates to the use of said cell composition and a method for preparing radiolabeled mesenchymal stem cells.

### BACKGROUND

Science and industry use radioisotopes in a variety of ways to gain information that cannot be obtained in any other way. Nuclear medicine uses radiation to provide diagnostic information about the functioning of a person's specific organs, to develop new therapies, or to treat them. One of the many radioisotopes is technetium-99m (^{99m}Tc), which is routinely used in nuclear medicine. The advantage of ^{99m}Tc is, amongst others, the ease of production through the transportable ⁹⁹Mo/^{99m}Tc generator, which constitutes a very efficient and reliable source of high specific activity.

Several biomolecules were labeled with ^{99m}Tc for medical imaging. Cell-labeling strategies have relied on transport of a radiometal (¹¹¹In, ^{99m}Tc, ⁶⁴Cu, ⁸⁹Zr) into cells in conjunction with chelators like, oxine, hexamethylpropyleneamine oxime (HMPAO), pyruvaldehyde-bis(N₄-methylthiosemicarbazone) (PTSM) or protamine sulfate. However, preparation of chelators generally requires multistep synthesis, and their incorporation into biomolecules often lacks efficiency and is further complicated by cross-reactivity with other functional groups present.

EP 3 265 134 discloses methods of ex vivo labeling of a biological material for in vivo imaging, methods of labeling a biological material in vivo, methods for preparing a labeling agent, and methods for in vivo imaging of a subject using a biological material labeled with a labeling agent.

BRPI0704971 discloses marking of leukocytes and stem cells with radionuclides, especially technetium-99m. The labeled cells are used for subsequent in vitro or in vivo application in the evaluation of inflammatory and/or infectious processes and in vivo monitoring of cell therapy.

With a growing interest in cell-based therapies, also stem cells were deployed. Mesenchymal stem cells (MSCs) are labeled with ^{99m}Tc-HMPAO. However, the labeling efficiency and post-labeling cell viability of the MSCs is low, as disclosed in Patel et al. 2018, Trela et al. 2014, and Spriet et al. 2014. Further, Adam G Laing et al. 2018 also describes human MSCs in vitro incubated with ^{99m}Tc, and Tobias L. Grossner et al. 2019 discloses to human MSCs in vitro incubated with ^{99m}Tc-hydroxydiphosphonate.

Accordingly, there remains a need in the art for an improved cell-labeling method, wherein the method scores a high labeling efficiency and wherein the labeled cells have a high post-labeling cell viability. In addition, the method should be efficient and cost-effective. The present invention aims to resolve at least some of the problems and disadvantages mentioned above.

### SUMMARY OF THE INVENTION

The present invention and embodiments thereof serve to provide a solution to one or more of above-mentioned disadvantages. To this end, the present invention provides for a cell composition according to claim 1. Preferred embodiments of the cell composition are shown in any of the claims 2 to 7.

In a second aspect, the present invention relates to a use according to claim 8, claim 9, claim 10 and claim 11. A preferred embodiment of the use is claim 12.

In a third aspect the present invention relates to a method for preparing radiolabeled mesenchymal stem cells according to claim 13. Preferred embodiments of the method are shown in any of the claims 14 to 17.

### FIGURES

FIG. 1 shows the measured radioactivity 10 minutes, 60 minutes, 6 hours and 24 hours following intravenous (IV), intramuscular (IM) and subcutaneous (SC) injection of the control product in dogs.

FIG. 2 shows the measured radioactivity 10 minutes, 60 minutes, 6 hours and 24 hours following intravenous (normal dose: IV, higher dose: IV+), intramuscular (IM) and subcutaneous (SC) injection of radiolabeled equine peripheral blood-derived-MSCs in dogs.

FIG. 3 shows the evolution over time in the heart, lung, liver and bladder for each dog following intravenous injection of free ^{99m}Tc (black) and radiolabeled ePB-MSCs (grey).

FIG. 4 shows a scintigraphic image of the lungs (left) and kidneys (right) of a horse 1 h post-injection with ^{99m}Tc labelled ePB-MSCs.

FIGS. 5a and 5b shows the relative uptake of free ^{99m}Tc and ^{99m}Tc labelled ePB-MSCs in different organs of interest of horses at 1 h and 6 h post-injection.

FIG. 6 shows (top) a dorsal view of the radioactive distribution in the kidneys of a horse 1 h (left) and 6 h (right) post-injection with the ^{99m}Tc labelled ePB-MSCs; and shows (bottom) a left lateral view of the radioactive distribution in the stomach of a horse 1 h (left) and 6 h (right) post-injection with free ^{99m}Tc.

### DETAILED DESCRIPTION OF THE INVENTION

The present invention concerns a cell composition comprising isolated radiolabeled mesenchymal stem cells (MSCs) with technetium-99m (^{99m}Tc), wherein said radiolabel is essentially free of hexamethylpropylene amine oxime (HMPAO). Present invention further relates to the use of said cell composition and a method for radiolabeling MSCs with ^{99m}Tc.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of ±20% or less, preferably ±10% or less, more preferably ±5% or less, even more preferably ±1% or less, and still more preferably ±0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, or ≥7 etc. of said members, and up to all said members.

The term “post-labeling cell viability” in current invention, refers to the cell viability of the cells after labeling, wherein cell viability is within 24 h after labeling and does not include any further culturing steps of the labeled cells. Preferably the cell viability is assessed within 12 h after labeling, more preferably within 6 h, 5 h, 4 h, 3 h, 2 h, 1.5 h after labeling. Desirably, the cell viability is assessed within the first hour after labeling, more desirably within 50 min, 40 min, 30 min, 20 min, 10 min, 5 min, 2 min, most desirable immediately after labeling.

The term “substantially free of” or “essentially free of” in current invention refers to the exclusion of HMPAO, or any other equivalent of HMPAO, from the radiolabeled composition. Specifically, HMPAO is present in an amount of less than about 10 parts per million (ppm), more specifically less than or equal to about 1 ppm, less than or equal to about 0.1 ppm, less than or equal to about 0.01 ppm, and more specifically less than or equal to about 0.001 ppm, based on the total weight of all components used in the composition.

The term “label persistence” in current invention, refers to a stable integration of the radiolabel in and/or on the MSCs in vitro and in vivo. Preferably, at most 10% of the radiolabel disintegrates from the MSCs, more preferably at most 5%, more preferably at most 2%, more preferably at most 1%, more preferably at most 0.1% disintegrates from the MSCs within 1 h, 2 h, 3 h, 4 h, 5 h, 6 h, even 24 h after the labeling was established.

In a first aspect, the invention relates to a cell composition comprising isolated MSCs, wherein said MSCs are radiolabeled with ^{99m}Tc (^{99m}Tc-MSCs), wherein said radiolabel is essentially free of HMPAO, and wherein said ^{99m}Tc-MSCs have a post-labeling cell viability of at least 55%.

Technetium-99m (“m” indicates that this is a metastable nuclear isomer) is used in radioactive isotope medical sciences. For example, ^{99m}Tc is a radioactive tracer for medical imaging that tracks in the body. It is well suited to the role because it emits readily detectable 140 keV gamma rays, and its half-life is 6.01 hours (meaning that about 94% of it decays to technetium-99m in 24 hours). The chemistry of technetium allows it to be bound to a variety of biochemical compounds, each of which determines how it is metabolized and deposited in the body, and this single isotope can be used for a multitude imaging tests and functional studies of the brain, heart muscle, thyroid, lungs, liver, gall bladder, kidneys, skeleton, blood, and tumors.

HMPAO is a widely used radiopharmaceutical, which is used as a chelating agent for the radioisotope technetium-99m in cell labeling. However, HMPAO may result in adverse cytotoxic advents. In addition, the inventors have found that the labeling efficiency and sample viability was higher in MSCs in deficiency of HMPAO, of which the results are shown in example 4.

It was found by the inventors that by radiolabeling MSCs with ^{99m}Tc, wherein said radiolabel is essentially free of HMPAO, the ^{99m}Tc-MSCs have a higher post-labeling cell viability than ^{99m}Tc-HMPAO-MSCs. Post-labeling cell viability of at least 55% is advantageous in that the cell composition comprises a high amount of viable ^{99m}Tc-MSCs. Loss of cell viability of post-labeled MSCs is avoidable for many reasons, not in the least due to the costly procedure of obtaining cells and labeling the latter. In addition, low cell viability limits subsequent use of the cells. The current invention solves this problem.

In addition, because the labeling does not require the use of HMPAO, the procedure is considerably lower in cost, as HMPAO kits are generally expensive.

Said ^{99m}Tc-MSCs of the cell composition of current invention have, next to the high cell viability, a good label persistence at 6 h. Preferably the radiolabel persistence of said ^{99m}Tc-MSCs is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, more preferably at least 99%, more preferably at least 99.9% at 6 hours. A lack of label persistence may complicate the interpretation of the apparent redistribution of the radioactivity over time. For instance, presence of urinary radioactivity indicates towards dissociation of the label from the MSCs, since MSCs do not filter through the kidneys.

Preferably, the cell composition is essentially free of a chelator or equivalent of HMPAO. This not only results in an easier and cost-effective labeling method, it also lowers the toxicity of the resulting cell composition.

In a preferred embodiment of the invention, the ^{99m}Tc is reduced by means of stannous chloride towards technetium-99m stannous chloride (^{99m}Tc—SnCl₂).

The minimal concentrations of stannous chloride in the cell composition of current invention restrain any toxic effects of tin. Furthermore, the biological activity of the cell composition is not affected by the presence of ^{99m}Tc—SnCl₂.

In a more preferred embodiment of the invention, the post-labeling cell viability of said radiolabeled MSCs is at least 60%.

Preferably the post-labeling cell viability of said ^{99m}Tc-MSCs is at least 65%, more preferably at least 70%, more preferably at least 75%, more preferably at least 80%, more preferably at least 82%, more preferably at least 84%, more preferably at least 86%, more preferably at least 88%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%. Preferably the post-labeling cell viability of said ^{99m}Tc-MSCs is of between 65 and 99%, more preferably between 70 and 98%, more preferably between 75 and 97%, more preferably between 80 and 96%, more preferably between 82 and 95%, more preferably between 84 and 94%, more preferably between 86 and 93%, more preferably between 88 and 92%.

Cell viability is an important parameter in cell culturing and engineering to evaluate long-term survival of cells. Numerous, adequate assays are available to determine cell viability and are known by a skilled person. Different dyes or stainings differentiating living from dead cells, such as calcein AM (CaAM) or trypan blue (TB), are at hand. Cell counters, fluorescent microscopes and flow cytometry may be used to count the dyed cells.

In a particularly preferred embodiment, the cell composition of the invention comprises at least 60% radiolabeled MSCs, preferably between 65 and 95%, preferably between 75 and 95%, preferably between 85 and 95% radiolabeled MSCs. Preferably, the cell composition of current invention comprises at most 5% unlabeled MSCs, more preferably at most 4%, more preferably at most 3%, more preferably at most 2%, more preferably at most 1% unlabeled MSCs. Particularly, the cell composition of current invention comprises at most 5% unbound radiolabel, more particularly at most 4%, more preferably at most 3%, more preferably at most 2%, more preferably at most 1% unbound radiolabel.

High concentrations of unlabeled MSCs and/or unbound radiolabel indicate to low labeling efficiency, which should be avoided. Furthermore high concentrations of unlabeled MSCs and/or unbound radiolabel cause biological interference and should be avoided.

MSCs are considered to be a promising source of cells in regenerative medicine. They have large potential to differentiate into various tissue-specific populations and may be isolated from diverse tissues in desired quantities. As cells of potential autologous origin, they allow recipients to avoid the alloantigen responses. They also have the ability to create immunomodulatory microenvironment, and thus help to minimize organ damage caused by the inflammation and cells activated by the immune system. Furthermore, MSCs have migratory abilities, that is, they specifically migrate to the sites of inflammation and tissue damage which is typically associated with cytokine outburst.

The MSCs of the current invention may be isolated by any standard protocol known in the art. Said MSCs may be isolated from for instance bone marrow, (peripheral) blood, adipose tissue, neonatal birth-associated tissues including placenta (PL), umbilical cord (blood or tissue), amnion fluid, dermis, etc. The nature of the cells obtained through this method can be ascertained by means of markers, specific for mesenchymal stem cells. Preferably, markers are selected from the group consisting of vimentin, fibronectin, Ki67, or any combination thereof. As such the purity of the obtained cell populations can be analyzed, and the percentage of mesenchymal stem cells determined. The MSCs may be isolated by the method as described in BE 1 020 480.

In one embodiment, said MSCs are native MSCs. In an embodiment of current invention, said MSCs are differentiated into a specific cell lineage. In another embodiment of current invention, said MSCs are genetically manipulated.

In an embodiment of current invention, said MSCs are preferably from mammalian origin, preferably of human, equine, canine, or feline origin, most preferably equine origin.

In an embodiment, the stem cells used in the present invention are isolated from the blood of mammals, more preferably, from peripheral blood. By preference the used blood will originate from human, cat, dog or horse, most preferably equine derived.

In a possible embodiment, blood from a donor was used who was later also recipient of his isolated mesenchymal stem cells. In another embodiment, blood is used from donors in which the donor is preferably of the same family, gender or race as the recipient of the mesenchymal stem cells isolated from the blood of donors. In particular, these donors will be tested on common current transmittable diseases or pathologies, in order to avoid the risk of horizontal transmission of these pathologies or diseases through the stem cells.

In a preferred embodiment of current invention said cell composition further comprises one or more pharmaceutical active ingredients. In other embodiments, said cell composition may be the only component of the composition; hence in such embodiment the composition may consist of or consist essentially of ^{99m}Tc—SnCl₂-MSCs.

The pharmaceutical active ingredient is selected form the group comprising of anti-inflammatory agents, growth factors, cytokines, antibiotics, or other active and ancillary agents. These pharmaceutically active ingredients are selected in that they do not interfere with the post-labeling cell viability of the MSCs, the label persistence and label stability.

In one embodiment, the cell composition comprises the anti-inflammatory agent. Anti-inflammatory agents include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

In a preferred embodiment of current invention, said cell composition is formulated for administering in a patient, preferably via intravenous, intraarticular, subcutaneous, intramuscular, or intralesional administration.

These routes of administration will depend heavily on the desired application of radiolabeled mesenchymal stem cells and/or their differentiated form. The administration route is also relevant for MSCs to exert the immunosuppressive activity in vivo. The skilled person is able to decide the type of administration based on the application and differentiation state of the cells within the cell composition.

Thus, allogeneic MSCs have demonstrated therapeutic efficacy and pose advantages versus autologous cells in terms of processing, quantity, and control. The cell composition is preferably injected, therefore it is sterile, non-pyrogenic and non-toxic.

The present composition may comprise, in addition to the herein particularly specified active pharmaceutical ingredients, one or more pharmaceutical excipients. Suitable excipients depend on the dosage form and identities of said ingredients and can be selected by the skilled person. Pharmaceutical excipients should not interfere with the cell activity, cell viability, radiolabel stability, nor radiolabel persistence of ^{99m}Tc-MSCs or said pharmaceutical active ingredients.

The composition as described above in any of the embodiments may be combined with unlabeled isolated MSCs or stem cells for therapeutic purpose such as the treatment or prevention of a disease. In an embodiment, said unlabeled MSCs may be the same kind of MSCs as the labeled MSCs. In another embodiment, said MSCs may be of a different origin, such as a different tissue or a different donor. Such MSCs may be derived or isolated from, for instance, bone marrow, (peripheral) blood, adipose tissue, neonatal birth-associated tissues including placenta, umbilical cord (blood or tissue), amnion fluid, dermis, liver tissue, lung tissue, mammary glands, etc. In many cases, MSCs are derived from blood as this is less invasive, and simple and safe to collect. Said MSCs may be from any donor origin, such as mammalian origin. As non-limitative examples, this may be of human, equine, canine, or feline origin. In particular, these donors will be tested on common current transmittable diseases or pathologies, in order to avoid the risk of horizontal transmission of these pathologies or diseases through the stem cells. Said MSCs may be any of the MSCs as described above in any of the embodiments. Optionally or in a further embodiment, said composition may also comprise one or more pharmaceutical agents. By preference, said agents should not interfere with the cell activity, cell viability, radiolabel stability, nor radiolabel persistence of ^{99m}Tc-MSCs or the cell activity or cell viability of unlabeled MSCs. Without wishing to be limitative, said agents may include chemicals, proteins, DNA, RNA and small molecules.

The cell composition according to the present invention has very broad applicability. In general, MSCs are the focus of research for musculoskeletal injuries or diseases and of research for liver injuries and/or diseases, as MSCs meet many requirements for their use in cell therapies. MSCs display immune modulation and promotion of tissue regeneration properties. The benefits of cell-based therapies, in particular MSC-based therapies for, for instance osteoarthritis, is predominantly attributed to the anti-inflammatory and immunosuppressive actions of the MSCs. MSCs, exert immunomodulatory activities through multiple pathways that primarily result in inhibition of both innate and adaptive immune responses. In particular, MSCs suppress proliferation and cytokine release of both CD4⁺ and CD8⁺ T lymphocytes in an MHC-independent manner, as well as promote a shift from Th1/Th17 toward Th2 phenotype and the generation of regulatory T cells. An inhibitory effect on B cells, NK cells, monocytes/macrophages, dendritic cells, and neutrophils has also been reported.

Therefore in a second aspect, current invention relates to the cell composition for use in biodistribution.

The radiolabeled MSCs of current invention are optimal to observe biodistribution patterns in a subject. Thereby radiolabeled cells can be monitored in vivo and an overview of how and when the cells migrate through the subject can be established. The radiolabeled MSCs easily overt to the different organs in the body. The radiolabeled MSCs of current invention advantageously do not migrate to skeletal structures. For example, in case of injury, the radiolabeled cells may be administered and via the observation of the biodistribution patterns, the specific site of injury can be localized.

Radiolabeled cells can be in vivo localized with scintigraphy. After administration, photographing is effected by the use of a gamma camera or a scintillation scanner to obtain a scintigram. Also positron emission tomography (PET) or single photon emission computed tomography (SPECT) can be utilized to track and localize the radiolabeled cells.

In vivo biodistribution of MSCs is an important parameter to take into account when evaluating the efficacy of cell-based therapy with MSCs. After intravenous administration, the infused MSCs accumulate preferential in the lungs and this is generally accompanied by a transient immunomodulatory effect due to their short-term persistence in vivo regardless of their origin. This, obviously, might impact the bioavailability of the cells, their targeting to tissues, and their long-term therapeutic efficacy.

Preferably, current invention also relates to the cell composition for use in diagnostics.

The cell composition of current invention, once administered to the patient, can localize to specific organs or cells allowing imaging the extent of a disease-process in the body, based on the cellular function and physiology, rather than relying on physical changes in the tissue anatomy.

The involvement of MSCs in many physiological or physiopathological aspects offers the possibility of targeting these cells, and their related molecular products, to obtain an early diagnosis by a non-invasive approach.

Furthermore, current invention relates to the cell composition for use in cell-based therapeutics.

In addition to biodistribution and diagnostics, the cell composition of current invention may be used in cell-based therapeutics. The immunomodulatory properties of the MSCs of current invention have therapeutic capacities. Furthermore, the toxicity of tin compounds is related to the chemical form and because of the low doses of tin applied in ^{99m}Tc labeling, no toxic effects are present. Moreover, the radiolabeling of the MSCs does not interfere with the biological characteristics of the MSCs in the cell composition.

Preferably, the pharmaceutical active ingredients and excipients in the cell composition are selected in that they improve the therapy.

As mentioned above, the composition of the current invention may include unlabeled MSCs and/or pharmaceutical agents.

One of ordinary skill in the art would recognize that multiple administrations of the compositions of the invention may be required to effect the desired therapy. For example the cell composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Preferably, the cell composition of current invention may be used in musculoskeletal injuries and/or diseases. Non-limiting examples are tendon injuries, musculoskeletal diseases are osteoarthritis.

In another embodiment, the cell composition of the current invention may be used in the prevention and/or treatment of liver injuries and/or diseases. Non-limiting examples are acute liver injuries, hepatocutaneous syndrome, chronic liver diseases, acute or chronic liver failure, acute-on-chronic liver failure, non-cirrhotic chronic liver disease, cirrhosis, compensated cirrhosis, decompensated cirrhosis (DC), acute decompensated cirrhosis and acute decompensation (AD), pediatric liver diseases such as biliary atresia, drug-induced liver diseases, fatty liver or other fibrotic liver disease, nonalcoholic fatty liver diseases such as non-alcoholic fatty liver or non-alcoholic steatohepatitis, inborn errors of liver metabolism, inherited Blood Coagulation Disorder, progressive familial intrahepatic cholestasis type 1/2/3, alpha 1-Antitrypsin Deficiency, defect of liver cell transporters, Porphyria, primary biliary cirrhosis, sclerosing cholangitis, and liver degenerative disease.

It is obvious that the cell composition according to present invention equally relates to a composition for the manufacture of a medicament for use in cell-based therapies, for treating and/or preventing musculoskeletal injuries and/or diseases and for treating and/or preventing liver injuries and/or diseases, and for all embodiments as described above and below.

In a preferred embodiment of the second aspect, the composition is administered to a patient, preferably intravenous, intraarticular, subcutaneous, intramuscular, or intralesional.

In one embodiment, the amount of ^{99m}Tc-MSCs in the cell composition administered to a subject is at least 1×10⁴ cells, at least 2.5×10⁴ cells, at least 5×10⁴ cells, at least 7.5×10⁴ cells, at least 1×10⁵ cells, at least 2×10⁵ cell, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 1.1×10⁶ cells, at least 1.2×10⁶ cells, at least 1.3×10⁶ cells, at least 1.4×10⁶ cells, at least 1.5×10⁶ cells, at least 1.6×10⁶ cells, at least 1.7×10⁶ cells, at least 1.8×10⁶ cells, at least 1.9×10⁶ cells, at least 2×10⁶ cells, at least 2.1×10⁶ cells, at least 2.2×10⁶ cells, at least 2.3×10⁶ cells, at least 2.4×10⁶ cells, at least 2.5×10⁶ cells, at least 2.6×10⁶ cells, at least 2.7×10⁶ cells, at least 2.8×10⁶ cells, at least 2.9×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, at least 1×10⁷ cells.

In particular embodiments, about 1×10^{4 99m}Tc-MSCs to about 1×10^{7 99m}Tc-MSCs, about 2.5×10^{4 99m}Tc-MSCs to about 5×10^{6 99m}Tc-MSCs, about 5×10^{4 99m}Tc-MSCs to about 2.5×10^{6 99m}Tc-MSCs, about 7.5×10^{4 99m}Tc-MSCs to about 2.2×10^{6 99m}Tc-MSCs, about 1×10^{5 99m}Tc-MSCs to about 2×10^{6 99m}Tc-MSCs, about 2×10^{5 99m}Tc-MSCs to about 1.9×10^{6 99m}Tc-MSCs, about 3×10^{5 99m}Tc-MSCs to about 1.8×10^{6 99m}Tc-MSCs, about 4×10^{5 99m}Tc-MSCs to about 1.7×10^{6 99m}Tc-MSCs, about 5×10^{5 99m}Tc-MSCs to about 1.6×10^{6 99m}Tc-MSCs, about 6×10^{5 99m}Tc-MSCs to about 1.5×10^{6 99m}Tc-MSCs, about 7×10^{5 99m}Tc-MSCs to about 1.4×10^{6 99m}Tc-MSCs, about 8×10^{5 99m}Tc-MSCs to about 1.3×10^{6 99m}Tc-MSCs, about 9×10^{5 99m}Tc-MSCs to about 1.2×10^{9 99m}Tc-MSCs, or about 1×10^{6 99m}Tc-MSCs to about 1.1×10^{6 99m}Tc-MSCs are administered to a subject.

In one embodiment, the amount ^{99m}Tc-MSCs in the composition administered to a subject is at least 0.1×10⁴ cells/kg of bodyweight, preferably at least 0.5×10⁴ cells/kg of bodyweight, more preferably at least 1×10⁴ cells/kg of bodyweight, more preferably at least 2.5×10⁴ cells/kg of bodyweight, more preferably at least 5×10⁴ cells/kg of bodyweight, more preferably at least 7.5×10⁴ cells/kg of bodyweight, more preferably at least 1×10⁵ cells/kg of bodyweight, more preferably at least 2×10⁵ cells/kg of bodyweight, more preferably at least 3×10⁵ cells/kg of bodyweight, more preferably at least 4×10⁵ cells/kg of bodyweight, more preferably at least 5×10⁵ cells/kg of bodyweight, more preferably at least 6×10⁵ cells/kg of bodyweight, more preferably at least 7×10⁵ cells/kg of bodyweight, more preferably at least 8×10⁵ cells/kg of bodyweight, more preferably at least 9×10⁵ cells/kg of bodyweight, more preferably at least 1×10⁶ cells/kg of bodyweight, more preferably at least 2×10⁶ cells/kg of bodyweight, more preferably at least 3×10⁶ cells/kg of bodyweight, more preferably at least 4×10⁶ cells/kg of bodyweight, more preferably at least 5×10⁶ cells/kg of bodyweight, more preferably at least 1×10⁷ cells/kg of bodyweight.

In particular embodiments, about 1×10^{4 99m}Tc-MSCs/kg of bodyweight to about 1×10^{8 99m}Tc-MSCs/kg of bodyweight, about 0.5×10^{4 99m}Tc-MSCs/kg of bodyweight to about 1×10^{7 99m}Tc-MSCs/kg of bodyweight, about 1×10^{5 99m}Tc-MSCs/kg of bodyweight to about 5×10^{6 99m}Tc-MSCs/kg of bodyweight, about 2×10^{5 99m}Tc-MSCs/kg of bodyweight to about 2×10^{6 99m}Tc-MSCs/kg of bodyweight, about 3×10^{5 99m}Tc-MSCs/kg of bodyweight to about 1×10^{6 99m}Tc-MSCs/kg of bodyweight, or about 5×10^{5 99m}Tc-MSCs/kg of bodyweight to about 0.8×10^{6 99m}Tc-MSCs/kg of bodyweight are administered to a subject.

A third aspect of current invention is related to a method for preparing radiolabeled MSCs, wherein the method comprises the step of incubating a dose of ^{99m}Tc and a source of post-transition metal ions, preferably stannous ions, with MSCs in the presence of a buffering composition having a pH of between 7.5 and 9.5.

Preferably the buffering composition has a pH of between 7.6 and 9.4, more preferably, 7.7 and 9.3, more preferably 7.8 and 9.2, more preferably 7.9 and 9.1, and more preferably between 8 and 9.

This labeling method of current invention yields a highly stable radiolabeled product with high label persistence at 6 hours. Furthermore the conditions of said method allow for a high percentage of post-labeling cell viability of the labeled MSCs. Furthermore, the method of current invention is a simple, rapid and cheap procedure, without the need of a chelator, in that ^{99m}Tc is directly labeled with the MSCs.

Post-transition metal ions reducing ^{99m}Tc are very valuable, especially for biochemical compounds that do not contain disulfide bridges. In addition, said radiolabeling leaves the biological characteristics of the MSCs intact. Preferably, the post-transition metal ions are already dissolved in the buffering composition. The buffering composition is preferably a saturated aqueous solution of NaOH and NAHCO₃.

In a more preferred embodiment, said source of post-transition metal ions is a stannous chloride solution.

In accordance with the principles of this invention, the stannous ion is employed as the reducing agent, and in some embodiments a nonradioactive stannous chloride solution may be prepared in advance and stored indefinitely, until such time as the ^{99m}Tc is added. It is necessary to have the isotope present in a reduced state, that is, at less than the seven valence state.

Metallic reducing agents, like tin, are convenient and effective for ^{99m}Tc. Furthermore, only minimal concentrations of stannous chloride are necessary to reduce. These concentrations are advantageous as toxic effects of tin are restrained. ^{99m}Tc can be incorporated conveniently, economically, and with reliability into the MSCs. Preferably the concentration of stannous chloride is equal to or less than 5 μg/μl, more preferably equal to or less than 4.5 μg/μl, more preferably equal to or less than 4 μg/μl, more preferably equal to or less than 3.5 μg/μl, more preferably equal to or less than 3 μg/μl, more preferably equal to or less than 2.5 μg/μl, more preferably equal to or less than 2 μg/μl, more preferably equal to or less than 1.5 μg/μl, more preferably less equal to or than 1 μg/μl, more preferably less equal to or than 0.9 μg/μl, more preferably less equal to or than 0.8 μg/μl, more preferably less equal to or than 0.7 μg/μl, more preferably less equal to or than 0.6 μg/μl, more preferably equal to or less than 0.5 μg/μl.

The complex is prepared by mixing the MSCs ligand and sodium pertechnetate at room temperature using stannous chloride as the reducing agent.

Usually, the dose of ^{99m}Tc to be incubated varies upon the type of administration. In a particular embodiment of the method, the dose of ^{99m}Tc is between 10 and 150 mCi. Preferably the dose of ^{99m}Tc is between 12 and 145 mCi, more preferably between 14 and 140 mCi, more preferably between 16 and 135 mCi, more preferably between 18 and 130 mCi, more preferably between 20 and 125 mCi, more preferably between 21 and 120 mCi, more preferably between 22 and 115 mCi, more preferably between 23 and 110 mCi, more preferably between 24 and 105 mCi, more preferably between 24 and 100 mCi, more preferably between 25 and 100 mCi, more preferably between 25 and 96 mCi.

The doses of ^{99m}Tc are optimized to minimize the injection volume and ensure optimum solution concentrations.

In a preferred embodiment of the method, the step of incubating lasts for at most one hour at room temperature.

The term “room temperature” in current invention is to be understood as the temperature of about 18° C. to about 21° C. Incubation at lower or higher temperatures than room temperature leads to nonspecific binding of ^{99m}Tc with MSCs, and should preferably be avoided. Next to nonspecific binding, the post-labeling cell viability is affected by temperatures deviating from room temperature.

In a more preferred embodiment, the step of incubation preferably lasts for 15 to 45 minutes at room temperature.

Preferably, the step of incubation lasts for 17 to 43 min, more preferably for 19 to 41 min, more preferably for 21 to 39 min, more preferably for 23 to 37 min, more preferably to 25 to 35 min, more preferably 27 to 33 min, more preferably 28 to 32 min, more preferably 29 to 31 min.

The incubation step further improves the post-labeling cell viability of the radiolabeled MSCs of current invention.

In a further embodiment of the method, the method further comprises the step of removing at least an amount of unbound radiolabel.

Advantages of this labeling method are the rapid labeling method and the good post-labeling cell viability. Although the labeling efficiency is generally 85 to 90%, a post-labeling purification may be required to remove the unbound radiolabel and to achieve a radiochemical purity exceeding 95%.

Unbound radiolabel may be removed by use of for example a column, or by centrifugal force. MSCs are passed over a column resulting in several fractions. Radioactivity in all fractions is measured in a shielded well-type gamma counter. The radiolabeled MSCs elute in the fractions immediately after the void volume, and the unbound radiolabel elute in the later fractions. Fractions containing the labeled MSCs can be pooled and are ready for injection. To verify the radiochemical purity of the final preparation, the gel permeation chromatography procedure can be repeated with a trace amount of the pooled fractions. Preferably, said unbound radiolabel is reused.

The invention is further described by the following non-limiting examples which further illustrate the invention, and are not intended to, nor should they be interpreted to, limit the scope of the invention.

### EXAMPLES

The present invention will now be further exemplified with reference to the following examples. The present invention is in no way limited to the given.

### Example 1: Labeling Mesenchymal Stem Cells According to Current Invention

Mesenchymal stem cells are isolated from peripheral blood of horses prepared and cultured as described in WO 2014 053 420.

Stannous chloride solution is prepared by dissolving tin(II)chloride in water of pH 8.5, in that the solution has a concentration of 0.5 μg/μl tin(II)chloride. MSCs are thawed and suspended in expansion medium type II. 1×10⁶ cells are isolated and pelleted in said expansion medium. The 45 mCi ^{99m}Tc is admixed with the stannous chloride solution and MSCs in 0.9% saline solution. The admixture is incubated for 30 min at room temperature. After incubation the mixture is centrifuged at 500 g for 5 minutes. The supernatant is isolated, where after, the radioactivity of the pelleted cells and supernatant is measured. Followingly the cells are resuspended in 5 ml DMEM and are centrifuged at 500 g for 5 minutes. The supernatant is isolated, where after, the radioactivity of the pelleted cells and supernatant is once again measured. Next the pelleted cells are resuspended in 1 ml DMEM. The post-labeling cell viability (PLCV) and labeling efficiency (LE) was immediately determined after labeling.

The labeling efficiency was calculated form the radioactivity measured in the cell pellet and supernatant using the following formula: (cell activity/(cell activity+supernatant activity 1+supernatant activity 2))×100. Table 1 gives an overview of the PLCV and LE of the four different samples, which were prepared in duo.

TABLE 1
Overview of post-labeling cell viability and labeling
efficiency immediately after labeling

| | PLCV | LE |
|---|---|---|
| Sample 1 | 71.79% | 51.41% |
| | 87.95% | 71.50% |
| Sample 2 | 89.47% | 73.17% |
| | 88.52% | 59.58% |
| Sample 3 | 83.00% | 67.80% |
| | 55.00% | 83.80% |
| Sample 4 | 79.10% | 60.15% |
| | 66.66% | 62.90% |

The measure mean post-labeling cell viability was 77.22% and the mean labeling efficiency was 66.76%.

The labeling protocol can be used without problems on cells isolated from a different tissue from other mammals, such as human, dog or cat.

### Example 2: Comparison of ^{99m}Tc-HMPAO and ^{99m}Tc-SnCl₂ Labeled Mesenchymal Stem Cells

Mesenchymal stem cells originating form equine peripheral blood were labeled with ^{99m}Tc-HMPAO or ^{99m}Tc-SnCl₂, followed by an immediate assessment of the post-labeling cell viability and labeling efficiency.

The MSCs are thawed and suspended in expansion medium type II. 1×10⁶ cells are isolated and pelleted in said expansion medium. 45 mCi ^{99m}Tc was admixed with 125 μg/ml HMPAO solution, and incubated while rocking for 5 min at room temperature to allow HMPAO binding of ^{99m}Tc. The ^{99m}Tc-HMPAO label was admixed with the MSCs and further incubated under gentle rocking for 23 min at room temperature. The ^{99m}Tc-HMPAO-MSCs were then centrifuged at 200 g for 10 min, washed once with 5 ml sterile saline and centrifuged again. ^{99m}Tc-SnCl₂-MSCs were prepared as documented in previous example. Both labeling methods were repeated three times for comparison. The mean post-labeling cell viability and labeling efficiency of ^{99m}Tc-HMPAO-MSCs was respectively 37.31% and 29.18%. The mean post-labeling cell viability and labeling efficiency of ^{99m}Tc-SnCl₂-MSCs was respectively 94.47% and 87.26%. In addition, the labeling persistence of ^{99m}Tc-HMPAO-MSCs and ^{99m}Tc-SnCl₂-MSCs was assessed at 6 h after labeling, wherein the labeling persistence of ^{99m}Tc-HMPAO-MSCs was 54% and ^{99m}Tc-SnCl₂-MSCs was 83%.

### Example 3: Biodistribution of ^{99m}Tc-SnCl₂-MSCs

Dogs were positioned in the gamma camera for injections. About 300.000 to 800.000 radiolabeled MSCs were injected per dog. The radiolabeled MSCs were suspended in 5 ml of saline and injected slowly through a 20 g catheter in one of the cephalic veins. A gamma camera equipped with a low-energy all-purpose collimator and peak at 140 keV was used for the imaging. The whole body of the dog, except for the head, was included in the field of view.

The distribution of the radiolabeled MSCs was assessed subjectively through the whole body. Scintigraphic images of the dog immediately (T0), 1 h (T1), 6 h (T6), 12 h (T12), and 24 h (T24) after systemic injection of radiolabeled mesenchymal stem cells were acquired. A specific uptake of the radiolabeled MSCs in the organs was noted. A clear uptake of the radiolabeled MSCs in the heart, lung, liver, spleen, left kidney and bladder were recorded, see Table 2 below. Organs were counted for radioactivity uptake, and the data was expressed as absorbed radiation dose (mCi) and the percentage injected dose per gram tissue (% ID/g tissue or % ID).

TABLE 2
Overview of the uptake of radiolabeled MSCs in different organs

| | Heart | Lung | Liver | Spleen | Left kidney | Bladder |
|---|---|---|---|---|---|---|
| T0 | 0.35 mCi | 1.05 mCi | 4.16 mCi | 0.17 mCi | 0.14 mCi | 0.18 mCi |
| | 2.10 % ID | 6.46 % ID | 25.16 % ID | 1.01 % ID | 0.84 % ID | 1.06 % ID |
| T1 | 0.23 mCi | 0.78 mCi | 4.36 mCi | 0.16 mCi | 0.14 mCi | 0.63 mCi |
| | 1.38 % ID | 4.84 % ID | 26.69 % ID | 0.98 % ID | 0.87 % ID | 4.11 % ID |
| T6 | 0.04 mCi | 0.11 mCi | 1.26 mCi | 0.05 mCi | 0.04 mCi | 0.34 mCi |
| | 0.25 % ID | 0.64 % ID | 7.59 % ID | 0.30 % ID | 0.25 % ID | 1.98 % ID |
| T12 | 0.01 mCi | 0.02 mCi | 0.37 mCi | 0.01 mCi | 0.01 mCi | 0.02 mCi |
| | 0.05 % ID | 0.16 % ID | 2.31 % ID | 0.08 % ID | 0.09 % ID | 0.13 % ID |
| T24 | 0.00 mCi | 0.01 mCi | 0.11 mCi | 0.00 mCi | 0.00 mCi | 0.00 mCi |
| | 0.01 % ID | 0.04 %ID | 0.69 % ID | 0.01 % ID | 0.02 % ID | 0.01 % ID |

### Example 4: Biodistribution of ^{99m}Tc-SnCl₂-MSCs after Intravenous (IV), Intramuscular (IM) and Subcutaneous (SC) Administration

### Methods

### Experiments

Three different studies were performed. In a first study, the biodistribution of intravenously administrated equine peripheral blood-derived MSCs (ePB-MSCs) was evaluated in four dogs. In the second study, the biodistribution of intramuscularly and subcutaneously administrated ePB-MSCs was evaluated in four dogs. Finally, in a third study, the biodistribution of a higher dose of ePB-MSCs following intravenous administration was evaluated in three dogs.

### Animals

Four healthy adult research dogs were included in the two first studies; three dogs from the second study were re-used for the third study. All dogs were purpose bred adult beagles (16-23 months). Two males and two females were included in the first two studies and one male and two female dogs were included in the third study. The dogs were housed in groups of 2, in a pen of 4 by 4 by 2 m (L×W×H) so permanent visual, olfactorial, tactile and auditive contact between dogs was possible. Toys were provided for the dogs to play with in their pen. Cleaning of the dog pens was performed daily. The floor was covered with wood shavings to improve lying comfort for the dogs. The animals were let out in dog runs to play and run for minimal 1 hour a day. After the study this minimal time was increased depending on the need of the dogs.

A daily general physical assessment was performed for each study evaluating the following parameters: rectal temperature, respiratory rate, heart rate, mucosal membranes, capillary refill time, body conditions score, mentation and hydration.

### Control Product Preparation

For the preparation of the control product (CP), 20±5 millicurie (mCi) (740±185 megabecquerel (MBq)) of freshly eluted ^{99m}Tc Pertechnetate (99m Tc) from a molybdenum generator (GE health care, Eindhoven, The Netherlands) was added to 1 mL of Dulbecco's Modified Eagle low glucose Medium (DMEM) (Life Technologies Europe BV, Belgium).

Collection and Culture of ePB-MSCs

The ePB-MSCs were good manufacturing practices (GMP) manufactured in a GMP-certified site. Briefly, blood was taken from the jugular vein of a donor horse and the MSCs were isolated. The serum was analyzed for a range of transmittable diseases by Bose laboratory (Harsum, Germany). The blood was centrifuged and the buffy coat was collected for gradient centrifugation. After washing, the ePB-MSCs were cultivated until passage 5 and a characterization for viability, morphology, presence of cell surface markers and population doubling time was performed. Next the ePB-MSCs were frozen as an intermediate cell stock. When characterization was completed, the intermediate cell stock was thawed and cultivated until passage 10 before being trypsinezed, resuspended, filtered twice trough a 40 μm filter and vialed at 3×10⁵ cells/mL in a mixture of DMEM and 10% dimethylsulfoxide (DMSO). The vials were stored at −80° C. until further use.

^{99m} Tc-Labeling of the ePB-MSCs

The technique of ^{99m}Tc labeling the ePB-MSCs was based on an optimization study. First, stannous chloride powder (Sigma Aldrich, US) was dissolved in sterile basic water (pH 8.5). Next, 0.9×10⁶ ePB-MSCs were thawed in the hand palm, transferred into growth medium and centrifuged for pelleting. The cell pellet was then resuspended in 4 mL of saline and mixed with 5 μg SnCl₂ and 45±5 mCi (1665±185 MBq) of freshly eluted ^{99m}Tc from a molybdenum generator (GE health care, Eindhoven, The Netherlands). Next the preparation was incubated for 30 minutes at room temperature before being centrifuged. The cell pellet was washed with 5 mL DMEM and centrifuged again. The final cell pellet was resuspended in 1 mL of DMEM and the viability of the ePB-MSCs following the labeling was determined using trypan blue. The radioactivity of the supernatant was measured after each centrifugation step in a dosiscalibrator and used to calculate the labeling efficiency.

### Treatment

In the first study, each dog received two intravenous injections; first the dogs were injected with the control product: freshly eluted ^{99m}Tc dissolved in DMEM and at least 7 days later they received a second injection with ^{99m}Tc-labeled ePB-MSCs. For the second study, 4 injections were administered to each dog. The dogs first received an IM injection with the control product, next a SC with the control product, then an IM injection with the ^{99m}Tc-labeled ePB-MSCs and finally a SC administration of the ^{99m}Tc-labeled ePB-MSCs. At least 7 days separated each injection. In the third study, the three dogs received a single IV injection with ^{99m}Tc-labeled ePB-MSCs.

The dogs were put under general anesthesia and positioned in sternal recumbency on the gamma camera before each injection. To obtain general anesthesia, the dogs were first sedated with dexmedetomidine (12-25 μg/kg IM), next induction was obtained with propofol (dosage on effect) and anesthesia was maintained with isoflurane 1.2-1.4% (on effect) in 100% oxygen following endotracheal intubation. The intravenous injection was administered through a 22-gauge catheter in one of the cephalic veins, the intramuscular injection was performed in the left quadriceps muscle and the subcutaneous injection was administered at the back of the neck.

### Imaging Protocol

A two-headed gamma camera, equipped with low energy high resolution collimators (GCA 7200 A; Toshiba) was used for the scintigraphic investigation. The whole body scan was obtained with the detectors of the SPECT scanner moving simultaneous dorsally and ventrally from head to tail of the dog over 10 minutes. All dogs were kept under general anesthesia during all the acquisitions. For all 3 studies, data collection of the first hour consisted of 6 successive acquisitions of each 10 minutes. The start of the first acquisition was simultaneous with injection of the radioactive compound and the dog remained in the same position for all 6 scans. Next, in the first study, total body scans (each lasting 10 minutes) were performed at 2 h, 4 h, 8 h, 12 h, 24 h and 36 h after placebo control and labeled ePB-MSCs administration using propofol (dosage on effect). For the second and the third study, 6 successive 10 minutes' total body scans performed during the first hour after injection were followed by total body scans (each lasting 10 minutes) at 6 h and 24 h after each injection. For all studies, a syringe with a known amount of radioactivity to calculate % injected dose (ID), was simultaneous scanned with the dog. Care was taken for the dog's re-positioning on the table, to avoid too much spatial deviation on the scans following the first hour scans.

### Image Interpretation

First, the distribution of the placebo control and the labeled ePB-MSCs was assessed descriptively through the whole body. Consequently, the radioactivity was quantified in different manually drawn regions of interest (ROI) on the dorsal and ventral view of the whole body scans (matrix size 512×1024) using the free-hand region of interest tool of a DICOM viewing software platform (Hermes MultiModality™, Nuclear Diagnostics, Sweden). A geometric mean of dorsal and ventral activity for each time point and each ROI was calculated to compensate for attenuation. Relative uptake was expressed as % of decay corrected injected activity for each region of interest per time point and calculated based on the known standard activity. To keep shape and sizes (number of pixels) of the different organ ROI's uniform, a ROI template was created per study and per dog and used for the different time points. A specific organ ROI was drawn on the image on which the organ was best delineated and thereafter used for the other images. Due to minor positioning deviations in between scans, ROI's had to be replaced on some images, however without changing the shape and size.

Due to the low sample size of four animals, only the overall effects in the heart, lung, liver and bladder following intravenous injection were taken into account for statistical analysis. The data were analyzed with SAS® statistical analysis software (version 9.4, SAS Institute Inc., Cary, N.C., USA). For the intramuscular and subcutaneous injections no statistical analysis was performed since a high radioactivity uptake remained at the injection sites following the injections of the radiolabeled ePB-MSCs and only a descriptive evaluation seemed appropriate. The overall statistical difference between intravenous administration of the free 99m Tc and the radiolabeled ePB-MSCs in the heart, lungs, liver and bladder was calculated using the area under the curve (AUC). The AUC was calculated using the trapezoidal method and can be written as a weighted sum of the observations. To allow a better interpretation of the AUC, it was presented as the weighted mean of the observations, using the weights of the observations in the AUC sum. A paired t-test was performed for this AUC for each organ separately, using the dog as a block effect. The time effects were described descriptively. The normality distribution assumption of the residuals was tested using the Shapiro-Wilks test and could not be rejected.

### Results

### Control Product Preparation

The injected ^{99m}Tc activity for the CP injection in each dog is displayed in Table 3.

TABLE 3
^{99m}Tc activity and route of the CP injected to
each dog in the different studies

| Study | Dog | Injection Route | Injected ^{99m}Tc activity (mCi) |
|---|---|---|---|
| 1 | 1 | IV | 20.04 |
| | 2 | | 22.55 |
| | 3 | | 22.70 |
| | 4 | | 19.26 |
| 2 | 6 | IM | 18.60 |
| | 7 | | 25.00 |
| | 8 | | 22.45 |
| | 9 | | 21.24 |
| | 6 | SC | 18.15 |
| | 7 | | 21.50 |
| | 8 | | 20.60 |
| | 9 | | 18.96 |

mCi: millicurie,
IV: intravenous,
IM: intramuscular,
SC: subcutaneous

### Labeling Efficiency, Post-Labeling Viability and Injected Dose

For the first study, the mean (min-max) overall labeling efficiency was 64.76% (59.58-71.50%), the number of MSCs ranged between 305,000 and 415,000 and post-labeling cell viability amounted to 84.64% (79.10-88.52%). For the IM injection of the second study, a mean overall labeling efficiency of 69.37% (53.65-77.83%), 465,000 to 775,000 MSCs and post-labeling cell viability of 93.70% (90.32-96.96%) were obtained and for the SC administration of the same study, the mean overall labeling efficiency was 72.94% (66.21%-78.34%), the number of MSCs per sample was 730,000 to 825,000 and post-labeling cell viability was 94.96% (93.63-96.96%). Finally, a mean overall labeling efficiency of 82.50% (80.65-83.82%), 1,610,000 to 1,950,000 MSCs and a cell viability of 95.40% (93.17-97.44%) were obtained in the third study (Table 4).

TABLE 4
Injection route, 99 mTc activity, labeling efficiency, number
and viability of the labeled-ePB-MSCs injected to each dog
in the different studies

| | | | Injected | | | |
|---|---|---|---|---|---|---|
| | | | ^{99m}Tc | Labeling | Number | Viability |
| | | Injection | activity | efficiency | of | of |
| Study | Dog | Route | (mCi) | (%) | MSCs | MSCs (%) |
| 1 | 1 | IV | 17.36 | 71.50 | 415,000 | 87.95 |
| | 2 | | 17.64 | 59.58 | 305,000 | 88.52 |
| | 3 | | 19.40 | 67.80 | 385,000 | 83.00 |
| | 4 | | 16.38 | 60.15 | 335,000 | 79.10 |
| 2 | 5 | IM | 18.45 | 72.78 | 465,000 | 90.32 |
| | 6 | | 19.05 | 73.21 | 775,000 | 95.48 |
| | 7 | | 24.04 | 77.83 | 695,000 | 93.53 |
| | 8 | | 15.00 | 53.65 | 775,000 | 95.48 |
| | 5 | SC | 15.62 | 66.21 | 825,000 | 96.96 |
| | 6 | | 20.15 | 69.18 | 785,000 | 93.63 |
| | 7 | | 23.38 | 78.02 | 760,000 | 94.74 |
| | 8 | | 23.49 | 78.34 | 730,000 | 94.52 |
| 3 | 6 | IV | 19.56 | 80.65 | 1,810,000 | 95.58 |
| | 7 | | 27.85 | 83.04 | 1,950,000 | 97.44 |
| | 8 | | 21.37 | 83.82 | 1,610,000 | 93.17 |

mCi: millicurie, IV: intravenous, IM: intramuscular, SC: subcutaneous

### Safety

The parameters rectal temperature, respiratory rate, heart rate, mucosal membranes, capillary refill time, body conditions score, mentation and hydration were in the physiological range for all animals at all time points of observation. No abnormal general clinical signs were observed and no (serious) adverse events or suspected adverse drug reactions were observed during the study.

### Biodistribution

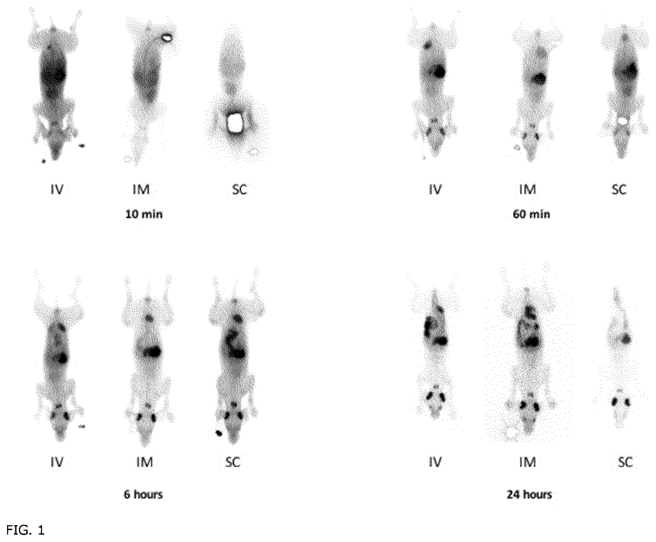

Intravenous injection of the CP led to an accumulation of free ^{99m}Tc in the following organs: heart, lung, liver, stomach, bladder, thyroid and salivary glands. Following intramuscular and subcutaneous administration of the placebo control, radioactivity uptake was seen in the heart, lung, stomach, bladder, thyroid and salivary glands. The highest uptake was seen in the stomach for all three injection routes with a progressive increase until 4 to 6 hours post-injection (Table 5, FIG. 1). In the first study, the scintigraphic examination 36 hours post-injection was performed, however the radioactive counts were too low to quantify. Therefore, this time point was not included for the evaluation of the biodistribution.

TABLE 5
Average percentage of injected dose observed in the different organs 10 minutes, 60 minutes,
6 hours and 24 hours following intravenous, intramuscular and subcutaneous injection of
the control product.

| | 10 min | | | 60 min | | | 6 hours | | | 24 hours | | |
|---|---|---|---|---|---|---|---|---|---|---|---|---|
| | IV | IM | SC | IV | IM | SC | IV | IM | SC | IV | IM | SC |
| Heart | 2.79 | 0.45 | 0.24 | 1.61 | 2.46 | 1.61 | 1.45 | 1.45 | 1.61 | 0.54 | 0.69 | 0.70 |
| Lung | 3.73 | 1.06 | 0.92 | 2.62 | 6.92 | 4.86 | 1.97 | 4.41 | 4.57 | 0.77 | 2.14 | 2.01 |
| Liver | 2.93 | / | / | 2.07 | / | / | 1.73 | / | / | 0.99 | / | / |
| Stomach | 4.84 | 1.62 | 0.53 | 9.77 | 17.36 | 9.24 | 8.98 | 17.25 | 14.37 | 5.02 | 9.36 | 7.32 |
| Bladder | 0.63 | 0.13 | 0.13 | 1.93 | 3.51 | 2.29 | 2.42 | 4.77 | 2.75 | 2.71 | 5.08 | 3.75 |
| Thyroid | 0.41 | 0.05 | 0.00 | 0.61 | 1.10 | 0.00 | 0.61 | 1.15 | 0.96 | 0.24 | 0.57 | 0.61 |
| Left | 0.33 | 0.02 | 0.12 | 0.77 | 1.03 | 0.45 | 1.31 | 1.84 | 1.44 | 1.34 | 2.47 | 2.67 |
| Salivary | | | | | | | | | | | | |
| Gland | | | | | | | | | | | | |
| Right | 0.33 | 0.01 | 0.13 | 0.72 | 0.99 | 0.45 | 0.88 | 1.60 | 1.33 | 0.98 | 2.09 | 2.81 |
| Salivary | | | | | | | | | | | | |
| Gland | | | | | | | | | | | | |
| Injection | / | 29.49 | 27.27 | / | 3.63 | 17.57 | / | 0.19 | 0.00 | / | 0.12 | 0.00 |
| Site | | | | | | | | | | | | |

IV: intravenous, IM: intramuscular, SC: subcutaneous

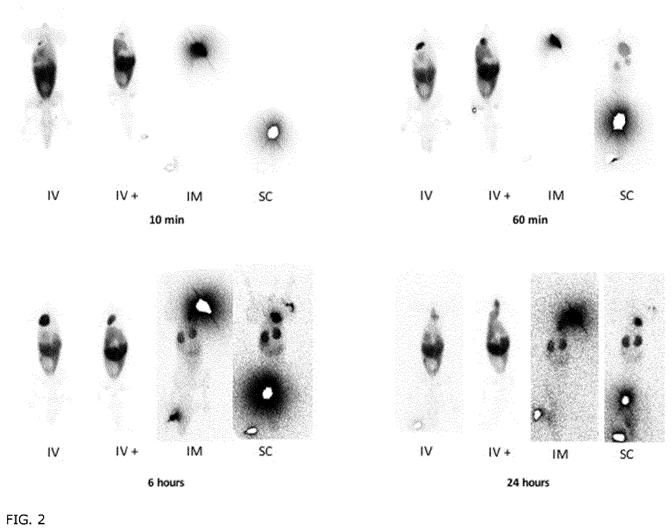

Following intravenous injections of the normal and the higher dose of the labeled ePB-MSCs into the cephalic vein of the dogs, presence was predominantly observed in the heart, lung, liver and bladder. Furthermore, minor radioactive uptake was seen in the spleen and kidneys. The highest uptake was seen in the liver with stable radioactivity until 24 hours post-injection. Intramuscular injection of the labeled ePB-MSCs led to a low radioactivity uptake in the following organs: lung, liver and kidneys. High uptake remained at the injection site for the entire evaluation period. This uptake at the injection site masked a potential uptake in the bladder. Finally, after subcutaneous injection of the labeled ePB-MSCs, low radioactivity uptake in the following organs was seen: kidneys and bladder. Again, high uptake remained at the injection site for the entire evaluation period. Radioactive uptake could be seen in the liver, however, this uptake was too low to be quantified. The uptake at the injection site masked a potential uptake in the heart and/or lung (Table 6, FIG. 2).

TABLE 6
Average percentage of injected dose observed in the different organs 10 minutes,
60 minutes, 6 hours and 24 to 36 hours following intravenous, intramuscular
and subcutaneous injection of the radiolabeled ePB-MSCs

| | | | | | | Left | | Injection |
|---|---|---|---|---|---|---|---|---|
| | | Heart | Lung | Liver | Spleen | Kidney | Bladder | Site |
| 10 | IV | 1.88 | 5.77 | 22.52 | 0.91 | 0.74 | 0.94 | / |
| min | IV+ | 2.00 | 10.64 | 22.38 | 1.58 | | 0.12 | / |
| | IM | / | 0.00 | / | / | 0.00 | / | 33.76 |
| | SC | / | / | / | / | 0.00 | 0.00 | 23.74 |
| 60 | IV | 1.19 | 3.63 | 23.27 | 0.87 | 0.66 | 3.49 | / |
| min | IV+ | 1.38 | 6.33 | 23.21 | 1.95 | | 1.08 | / |
| | IM | / | 0.03 | / | / | 0.06 | / | 31.08 |
| | SC | / | / | / | / | 0.03 | 0.28 | 23.73 |
| 6 | IV | 0.70 | 1.90 | 22.07 | 0.85 | 0.75 | 5.54 | / |
| hours | IV+ | 0.94 | 3.91 | 27.26 | 2.23 | | 1.28 | / |
| | IM | / | 0.00 | / | / | 0.08 | / | 40.42 |
| | SC | / | / | / | / | 0.06 | 23.57 | 14.67 |
| 24 | IV | 0.33 | 1.30 | 19.71 | 0.76 | 0.35 | 0.59 | / |
| hours | IV+ | 0.71 | 3.20 | 25.30 | 2.21 | | 1.67 | / |
| | IM | / | 0.00 | / | / | 0.09 | / | 44.95 |
| | SC | / | / | / | / | 0.22 | 28.02 | 35.07 |
| 36 | IV | 0.30 | 0.63 | 16.45 | 0.50 | 0.33 | 0.35 | / |
| hours | | | | | | | | |

IV: intravenous (normal dose: IV, higher dose: IV+), IM: intramuscular, SC: subcutaneous

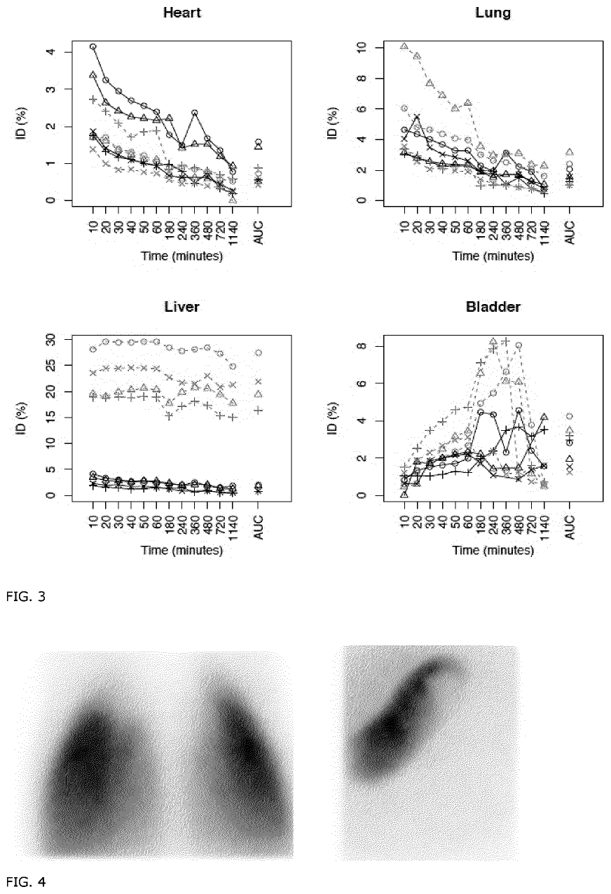

A significant difference (P-value=0.003) for ID % in the liver between the free ^{99m}Tc and radiolabeled ePB-MSCs could be found following IV administration. No significant difference was obtained following both IV injections in the heart (p=0.28), lung (p=0.58) or bladder (p=0.21) (FIG. 3).

### Discussion

The different studies describe the total body distribution of intravenously, intramuscularly and subcutaneously injected ^{99m}Tc labeled ePB-MSCs compared with free ^{99m}Tc during a 24 h follow-up period with scintigraphy in healthy dogs. To the authors' knowledge this is the first study comparing the biodistribution of ^{99m}Tc labeled ePB-MSCs with free ^{99m}Tc in dogs. Furthermore, a total body scintigraphy after intravenous, intramuscular and subcutaneous injection of ^{99m}Tc labeled ePB-MSCs has never been described.

The labeling efficiency and cell viability ranged between 59.58% and 83.82% and between 79.10% and 97.44% for all studies, respectively. This is considerably higher than the labeling efficiency of 42 to 57% reported by Spriet, Hunt et al. where the MSCs were labeled with 99m Tc-HMPAO. However, no post-labelling cell viability was reported for this study.

Free ^{99m}Tc is preferentially taken up by the stomach, thyroid gland and salivary glands. This was also seen in the current studies for the different injection routes (i.e. IM, SC and IV). No radioactive accumulation was observed in none of these organs at all time points following the different injections routes of ^{99m}Tc-labeled ePB-MSCs. Therefore, we could confirm the used ^{99m}Tc labeling technique resulted in a stable in vivo-complex with ePB-MSCs. Additionally, the labeling did not affect viability of the ePB-MSCs after injection, since it is assumed that cell death would cause a release of ^{99m}Tc since the cell membrane is no longer intact and accumulate in the aforementioned organs similar to free ^{99m}Tc injections. A lower uptake of free ^{99m}Tc was seen in the heart and lung following all injections and in the liver after intravenous injection. Finally, the previously described partial excretion route of ^{99m}Tc through glomerular filtration explains the increased uptake in the bladder.

No pronounced initial pulmonary trapping of the ePB-MSCs following the different injection routes was seen. In the third study, more ID % was detected in the lungs, however, this can be explained by the higher amount of cells injected. There was initial accumulation in the lungs after injecting the higher dose, indicating no long-term entrapment of the ePB-MSCs occurs after IV injection. In contrast, other groups using technetium-labeled mesenchymal stem cells described initial high pulmonary trapping. The absence of pulmonary entrapment in the current studies could be explained by the use of a different MSC source and a lower number of injected MSCs. In the study reported by Spriet et al., 10×10⁶ adipose tissue-derived MSCs were injected in the same dog breed as in our studies, whereas our group injected only 305,000 to 1,950,000 equine peripheral blood-derived MSCs in the dogs. Moreover, a part of the production process of the ePB-MSCs used in this study consists of a filtration process reducing the risk of cell clustering following the intravenous injection of the ePB-MSCs.

In contrast to the observations reported by Spriet, Hunt et al., a high liver uptake was seen following both intravenous injections (i.e. study 1 and study 3) of the ePB-MSCs which remained stable during the first 6 hours following the injection and only decreased slightly 24 hours post injection. These findings support the potential use of intravenously administered ePB-MSCs for the treatment of liver diseases such as acute liver injuries or hepatocutaneous syndrome.

Following IM and SC injection only a very low biodistribution of the radiolabeled ePB-MSCs was seen and a high amount of the injected MSCs stayed at the injection site throughout the 24-hour follow-up period. Consequently, the biodistribution of the ePB-MSCs following IM and SC injections appears to be different from intravenously injected ePB-MSCs.

The limitations of the studies were the absence of blinding and randomization. Blinding was not feasible because the wash out period of the radiolabeled ePB-MSCs is currently unknown and therefore could not be administered first before the free ^{99m}Tc This practical constraint together with the reported knowledge on distribution of free ^{99m}Tc in literature, would have meant that the investigator could have guessed with high certainty which animals would have received the free ^{99m}Tc and which ones the radiolabeled ePB-MSCs when evaluating the total body scans. However, the absence of blinding was mitigated by using an objective evaluation criterion for evaluation of biodistribution i.e. scintigraphic total body scans for quantifying radioactivity in a region of interest instead of using subjective scores. Another limitation was the low number of dogs included in the studies which limited the possibilities for statistical analysis.

### Conclusions

This study describes the biodistribution of radiolabeled ePB-MSCs following intravenous, intramuscular and subcutaneous injection in dogs measured by scintigraphic evaluation of radioactivity. During this study a distinct difference was noted in biodistribution of the radiolabeled ePB-MSCs and free ^{99m}Tc. This implies ePB-MSCs have a specific pharmacokinetic pattern after systemic administration in healthy animals. This study thus gives indications for more targeted sampling during safety studies. Additionally, it also provided information on the natural biodistribution pattern of the used ePB-MSCs which appeared to be different to previously reported experiments using different MSC sources.

### Example 5: Cytotoxic Effects of HMPAO on MSCs

MSCs were labeled with ^{99m}Tc in the presence of different amounts of HMPAO and/or SnCl₂. The labeling of MSCs with SnCl₂ or HMPAO is as discussed in respectively example 1 and 2. The labeling of MSCs with SnCl₂ and HMPAO, started by first admixing 45 mCi ^{99m}Tc with 125 μg/ml HMPAO solution, followed by 5 min incubation at room temperature while rocking, to subsequently admix the ^{99m}Tc-HMPAO with the stannous chloride solution and MSCs in 0.9% saline solution and further incubate under gentle rocking for 30 min at room temperature. After isolation of the labeled MSCs and resuspension in DMEM, the cell viability (PLCV) and labeling efficiency (LE) immediately after labeling was measured as described in table 7 below.

TABLE 7
Overview of cytotoxic effects on MSCs by measuring post-
labeling cell viability and labeling efficiency immediately
after labeling MSCs with HMPAO and/or SnCl₂.

| HMPAO addition | SnCl₂ addition | PLCV | LE |
|---|---|---|---|
| 3 mL | 0 μg | 67% | 2.9% |
| 3 mL | 0 μg | 78% | 0.7% |
| 4 mL | 15.2 μg | 3% | 7% |
| 2 mL | 7.6 μg | 79% | 6% |
| 2 mL | 7.6 μg | 8% | 31% |
| 0 mL | 7.6 μg | 84% | 40% |

Results clearly show a higher labeling efficiency and viability of MSCs in deficiency of HMPAO. And in case a high cell viability is obtained after ^{99m}Tc-HMPAO it is accompanied by a low labeling efficiency.

### Example 6: Biodistribution of ^{99m}Tc-Radiolabeled Equine Peripheral Blood-Derived MSCs after IV Injection in Healthy Horses

The goal of this study was to evaluate the biodistribution of intravenously (IV) injected ^{99m}Tc labelled equine peripheral blood derived MSCs (ePB-MSCs) with free IV injected ^{99m}Tc in healthy horses during a 6 hour follow-up period, using a full body scintigraphy imaging technique.

### Materials and Methods

### Animals

Four healthy adult horses (2 neutered male and 2 female horses, 7 to 19 years old) from two different breeds (2 Belgian warmblood and 2 French Trotter) were included in this study. All animals underwent a full veterinary examination before being included in the study and were stalled at least 2 days prior to the start of the study. During the study, the animals were housed in individual stables of 3.5 by 4 (L×W) according to normal housing procedures with water ad libitum and hay twice a day. During the study, a daily general clinical examination was performed (day 0-3). All horses received a total of two IV injections; the first consisted of free ^{99m}Tc dissolved in DMEM which served as control. The second injection was administered three days later and consisted of ^{99m}Tc-labelled ePB-MSC.

Collection and Culture of ePB-MSCs

ePB-MSCs were GMP-manufactured in a GMP-certified site. In short, all MSCs were isolated from venous blood, taken from the vena jugularis of one donor horse. Prior to culture, serum was examined on various transmittable diseases by Bose laboratory (Harsum, Germany). The ePB-MSCs were cultivated until passage 5 and thoroughly characterized for viability, morphology, presence of cell surface markers and population doubling time, before being frozen as an intermediate cell stock. After completion of the characterization, the intermediate cell stock was thawed and further cultivated until passage 10. Subsequently, ePB-MSCs were trypsinized, resuspended and vialed at 3×10⁵ cells/mL in a mixture of Dulbecco's Modified Eagle low glucose Medium (DMEM) and 10% dimethylsulfoxide (DMSO). Finally, the vials were stored at −80° C. until further use.

Optimizing the ^{99m}Tc-Labeling of ePB-MSCs

In order to find optimal conditions for the ^{99m}Tc labeling of ePB-MSCs, different reaction conditions have been tested and evaluated. All test conditions were performed with 740-1480 MBq of ^{99m}Tc (Drytec generator; supplier: GE, Eindhoven, The Netherlands) and with different amounts of ePB-MSCs (0.3×10⁶-1×10⁶-2×10⁶). Before labeling, the ePB-MSCs were thawed in the palm of a hand, dissolved in culture medium and centrifuged. Consequently, following reaction conditions were tested: free ^{99m}Tc with addition of 375 μg HMPAO, free ^{99m}Tc labeling with addition of 250 μg and 500 μg HMPAO combined with 7.6 μg and 15.2 μg tin (II) chloride (SnCl₂), respectively, and free ^{99m}Tc labeling with addition of solely SnCl₂ in different amounts (5 μg, 7.6 μg and 15.2 μg) in combination with saline. Basic water (pH 8.5) was used to dissolve the SnCl₂. Each reaction mixture was incubated at room temperature for 30 minutes while gently shaking the mixture every five minutes. After the incubation time, the cells were centrifuged and washed with DMEM before being centrifuged again. All supernatant was recovered in separate tubes. Finally, the cells were dissolved in 1 mL DMEM to obtain the final IV formulation. The labeling efficiency was calculated for each reaction condition and was defined as the cell pellet radioactivity divided by the cell pellet radioactivity+recovered supernatant. Finally, the post-labeling cell viability was determined using trypan blue. Finally, the optimal labeling condition was repeated in eightfold using 0.5±0.1 GBq ^{99m}Tc and 1.8±0.1 GBq ^{99m}Tc to further validate this technique.

^{99m}TC-Labeling of ePB-MSCs in Clinical Study

Optimizing labeling conditions were performed with relative low radioactivity of ^{99m}Tc and are intended to be used for feline and canine applications, respectively. However, in order to obtain enough counts on the 6 h post-injection scintigraphy in horses, upscaling of the ^{99m}Tc activity was needed for the current study. Therefore, the optimal labeling condition was selected and executed with an increased ^{99m}Tc activity of approximately 9.25 GBq and 20 μg of SnCl₂ instead of 5 μg. Prior to labeling, SnCl₂ powder (Sigma Aldrich, US) was dissolved in sterile basic water (pH 8.5) to obtain a solution of 0.5 mg/mL. Consequently, 1×10⁶ ePB-MSCs were thawed in the palm of a hand, transferred into growth medium and centrifuged for pelleting. The retained cell pellet was then resuspended in 4 mL NaCl and mixed with 20 μg SnCl₂ and 9.18±0.26 GBq of ^{99m}Tc (Drytec generator; supplier: GE, Eindhoven, The Netherlands). The mixture was incubated for 30 minutes at room temperature and gently shaken every 5 minutes after which it was centrifuged. Consequently, the cell pellet was washed with 5 mL DMEM and centrifuged again. The supernatant of both centrifuge steps was recovered for the calculation of the labeling efficiency. Finally, the cell pellet was resuspended in 1 mL of DMEM and the post-labelling viability was determined using trypan blue.

### Imaging Protocol

For the IV injections of the horses, a 18 G catheter was placed in the left jugular vein of each horse and sedation was achieved using detomidine hydrochloride (Domidine 10 mg/mL; Dechra, The Netherlands) 0.01 mg/kg body weight and butorphanol tartrate (butomidor 10 mg/mL; AST farma BV, Oudewater, Germany) 0.01 mg/kg body weight IV When required, detomidine hydrochloride (Domidine 10 mg/mL; Dechra) was added during the scan with a maximum of 0.002 mg/kg. The horses stood straight with all four limbs using a table to stabilize the head. On day 0, all four horses were IV injected with free ^{99m}Tc dissolved in DMEM. Consequently, on day 3, all horses were IV injected with ^{99m}Tc-labelled ePB-MSCs dissolved in DMEM as described above. Planar scintigraphy was performed 1 h and 6 h post-injection for both injections using a gamma camera (Equine HR, Medical Imaging Electronics) (matrix: 128×128; acquisition time: 60 seconds per time frame (60 frames); collimator: low energy high resolution (140 KeV, 15% symmetrical window); field of view: 600×410 mm). Following body parts were scanned: head, thorax and abdomen starting with the head, left thorax and left abdomen and continuing with the right abdomen and right thorax.

### Image Analysis

Distribution of the ^{99m}Tc labelled ePB-MSCs and free ^{99m}Tc was subjectively assessed through the whole body using Multimodality software (Nuclear diagnostics, Sweden). Different organs of interests were manually delineated for each horse at each time point: the lungs, bladder, stomach, kidney, parotid gland and thyroid. Radioactive counts per pixel were calculated for each organ of interest, decay corrected and divided by the initial injected radioactivity in the whole body to obtain a relative uptake value.

### Results

^{99m}Tc-Labeling of ePB-MSCs

In Table 8, an overview is given of the different labeling conditions tested, together with cell viability and labeling efficiency. The addition of various amounts of HMPAO to the reaction mixture yielded to very low labeling efficiencies. Furthermore, the combination of HMPAO with SnCl₂ had adverse effects on the cell viability of the ePB-MSCs. In contrast, adding only SnCl₂ to the reaction mixture had positive effect on the labeling efficiency with a maximum labeling efficiency of 74% and a maximum cell viability of 89% when using 1×10⁶ ePB-MSCs with 1.48 MBq ^{99m}Tc, together with 4 mL saline and 5 μg SnCl₂ (highlighted in the table with bold contours). Viability and labeling efficiency data of the experiment repeating the latter in eightfold is presented in Table 9. A mean viability of 70.1±10.9% and a labeling efficiency of 75.1±8.7% was found when using 0.5±0.1 GBq ^{99m}Tc to start. Using 1.8±0.1 GBq ^{99m}Tc for labeling, the ePB-MSCs resulted in a viability of 77.7±12.3% and labeling efficiency of 66.3±10.0%.

TABLE 8
An overview of the different labeling conditions with corresponding cell
viability and labeling efficiency. A: Addition of HMPAO and
HMPAO + SnCl2. B: Addition of SnCl2.

| | | | | | | |
|---|---|---|---|---|---|---|
| | | | | | Cell | Labeling |
| # ePB- | SnCl₂ | NaCl | ^{99m}TC | HMPAO | Viability | efficiency |
| MSCs | (μg) | (mL) | (GBq) | (μg) | (%) | (%) |

A

| | | | | | | |
|---|---|---|---|---|---|---|
| 3 × 10⁵ | / | / | 1.48 | 375 | 78 | 0.7 |
| 1 × 10⁶ | / | / | 1.48 | 375 | 67 | 2.9 |
| 1 × 10⁶ | 15.2 | / | 1.48 | 500 | 3 | 7 |
| 1 × 10⁶ | 7.6 | / | 0.74 | 250 | 79 | 6 |

B

| | | | | | | |
|---|---|---|---|---|---|---|
| 1 × 10⁶ | 15.2 | 4 | 1.48 | / | 45 | 61.8 |
| 1 × 10⁶ | 7.6 | 2 | 0.74 | / | 84 | 40 |
| 1 × 10⁶ | 5 | 4 | 1.48 | / | 71 | 74 |
| 1 × 10⁶ | 5 | 4 | 1.48 | / | 89 | 53 |
| 2 × 10⁶ | 5 | 4 | 1.48 | / | 77 | 46 |
| 2 × 10⁶ | 5 | 4 | 1.48 | / | 73 | 61 |

TABLE 9
Overview of the cell viability and labeling efficiency
after 99mTc labeling of the ePB-MSCs
using the optimal labeling condition.

| | 1.8 ± 0.1 GBq ^{99m}Tc | | 0.5 ± 0.1 GBq ^{99m}Tc | |
|---|---|---|---|---|
| | Cell | Labeling | Cell | Labeling |
| | Viability | efficiency | Viability | efficiency |
| # | (%) | (%) | (%) | (%) |
| 1 | 87.95 | 71.50 | 76.00 | 62.92 |
| 2 | 71.79 | 51.41 | 53.49 | 66.90 |
| 3 | 89.47 | 73.17 | 62.11 | 77.84 |
| 4 | 88.52 | 59.58 | 75.69 | 83.30 |
| 5 | 83.00 | 67.80 | 70.00 | 87.76 |
| 6 | 55.00 | 83.80 | 83.33 | 79.97 |
| 7 | 79.10 | 60.15 | 58.76 | 74.22 |
| 8 | 66.66 | 62.90 | 81.32 | 67.73 |
| Mean ± SD | 77.7 ± 12.3 | 66.3 ± 10.0 | 70.1 ± 10.9 | 75.1 ± 8.7 |

### Labeling Efficiency, Post-Labeling Viability and Injected Dose

During the clinical study, the mean (min-max) overall labeling efficiency was 55.7% (25.19-86.02%) (n=4) and post-labeling cell viability amounted to 72.4% (30.97-88.41%) (n=4) (see Table 10). Furthermore, the mean (min-max) IV injected dose per horse was 7.37 GBq (7.26-7.53 GBq) (n=4) for the free ^{99m}Tc and 3.92 GBq (2.09-5.88 GBq) (n=4) for the ^{99m}Tc-labelled ePB-MSCs. The total amount of injected ePB-MSCs varied from 0.35×10⁶ to max 0.61×10⁶. Finally, no physical-, behavioral abnormalities or adverse effects were reported during this clinical study.

TABLE 10
Overview of the cell viability and labeling
efficiency after 99mTc labeling of the ePB-
MSCs for the different horses of the clinical study.

| | Cell Viability (%) | Labeling efficiency (%) |
|---|---|---|
| Horse 1 | 88.41 | 86.02 |
| Horse 2 | 86.55 | 81.55 |
| Horse 3 | 30.97 | 29.84 |
| Horse 4 | 83.60 | 25.19 |

### Biodistribution

As described above, in three out of four horses a good labeling efficiency was present. However, because of the poor labeling efficiency and very low cell viability in horse 3, there were not enough counts during the scintigraphy examination at both time points. Therefore, results of horse 3 were assumed unreliable and therefore excluded from the analysis. Furthermore, on one scan, 1 h post-injection the free ^{99m}Tc, no image of the bladder could be retrieved.

When subjectively assessing the scans 1 h post-injection the free ^{99m}Tc, accumulation was predominantly observed in stomach, thyroid, bladder and parotid glands. On the ePB-MSC scans, an accumulation of ePB-MSCs was primarily seen in the lungs (FIG. 4) and kidneys. Due to superposition and attenuation, quantification of the liver and the spleen was not feasible. Furthermore, on the free ^{99m}Tc scans small activity was seen in the intestines, however, it was not possible to quantify the uptake. In addition, no accumulated radioactivity was seen in the intestines on the ePB-MSC scans. Finally, no radioactivity accumulation was found in the heart, although this could not be excluded due to superposition and attenuation. As it was not feasible to determine the exact percentage of accumulated radioactivity in each organ due to overlap and different camera positions for one organ, the relative radioactive uptake was compared between the two injections (free ^{99m}Tc vs. ePB-MSCs) and evaluated in function of the time for each organ of interest.

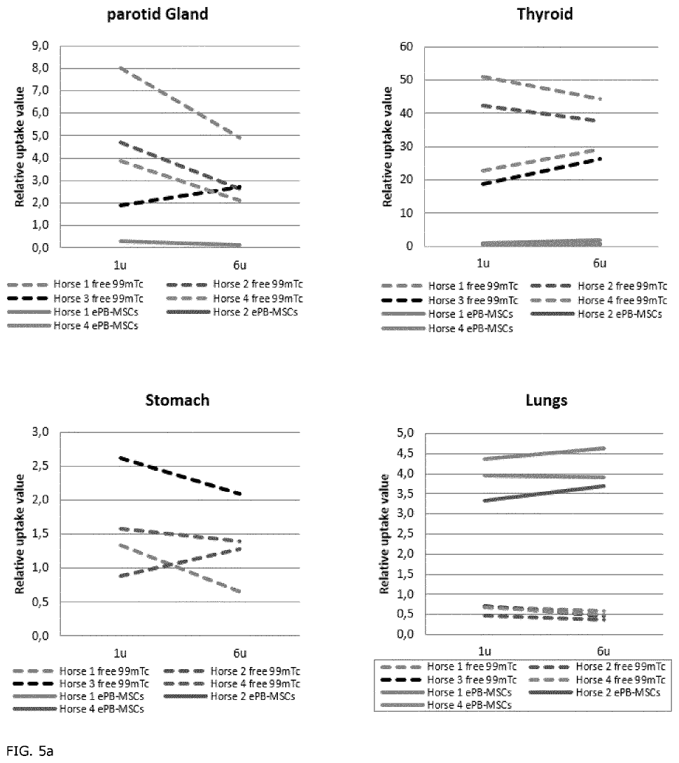

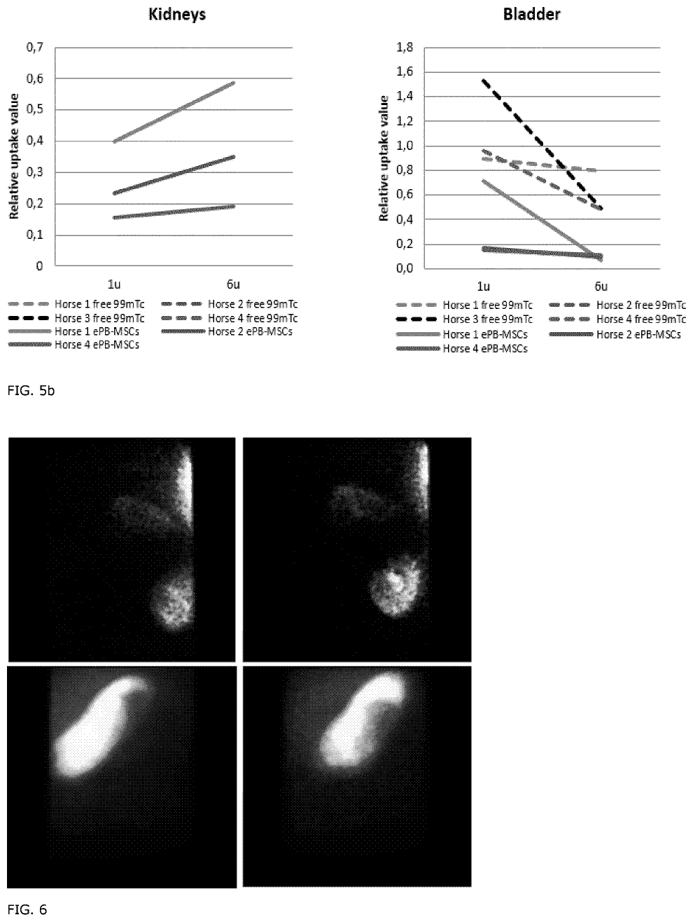

An overview of the biodistribution of free ^{99m}Tc and ^{99m}Tc labelled ePB-MSCs in all of the organs of interest is shown in FIGS. 5a and 5b (data not shown for individual horses on the graphs indicates no accumulation could be found in the organ of interest). High accumulation of free ^{99m}Tc was observed in the stomach on the 1 h post-injection scan and decreased in 3 out of 4 horses (75%) in function of the time (6 h post-injection scan). No accumulation of ^{99m}Tc labelled ePB-MSCs could be observed in the stomach at both time points. Furthermore, in contrast to the very low uptake of ^{99m}Tc labelled ePB-MSCs in the thyroid gland, a very high accumulation of free ^{99m}Tc was seen in the thyroid gland 1 h and 6 h after injection. Radioactive uptake increased after 6 h in 2 out of 4 horses while a decrease in the other two horses was seen. Similar results were found for the parotid glands, which showed high uptake of free ^{99m}Tc seen on the 1 h post-injection scan and decreased in 3 out of 4 horses (75%). A negligible uptake of ^{99m}Tc labelled ePB-MSCs was seen in the parotid glands, with uptake in only one of the three horses. A distinct radioactive uptake of free ^{99m}Tc and ^{99m}Tc labelled ePB-MSCs was seen in the lungs at both time points (FIG. 4). However, the uptake of ^{99m}Tc labelled ePB-MSCs was much higher compared to free ^{99m}Tc and increased in function of the time, which was not the case for the free ^{99m}Tc. In the kidneys accumulation of ^{99m}Tc labelled ePB-MSCs could be seen and increased in time, while no kidney accumulation after free ^{99m}Tc could be detected (FIG. 6). For both injections, radioactivity was found in the bladder of the horses, although accumulation was higher for free ^{99m}Tc. Nevertheless, a decreased uptake was found 6 h post-injection for both injections.

### DISCUSSION & CONCLUSION

This study demonstrates the total body distribution of ^{99m}Tc labelled ePB-MSCs compared with free ^{99m}Tc during a 6 h follow-up period with scintigraphy in healthy horses. To the authors' knowledge this is the first study comparing the biodistribution of ^{99m}Tc labelled ePB-MSCs with free ^{99m}Tc in horses. Furthermore, a total body scintigraphy after injecting ^{99m}Tc labelled ePB-MSCs has never been performed.

As the ^{99m}Tc labelling of ePB-MSCs has never been described before, several tests were conducted to find optimal conditions. Although, different studies have reported the successful use of HMPAO or the combination of HMPAO and SnCl₂ to label equine MSCs from different sources with ^{99m}Tc, this could not be replicated in the current study. All of the test conditions using different HMPAO conditions led to very low labeling efficiencies. However, adding 5 μg SnCl₂ to 1×10⁶ ePB-MSCs dissolved in 4 mL saline and 1.48 MBq ^{99m}Tc led to high labeling efficiency (53-74%) and cell viability (71-89%). This labeling condition was further tested and validated in eightfold and showed similar results with both 0.5±0.1 and 1.8±0.1 GBq ^{99m}Tc. Nevertheless, when scaling-up the radioactivity to 9.25 GBq and consequently increasing the SnCl₂ to 20 μg for equine use during the clinical phase of this study, labeling efficiencies variated between 25% and 86%, and were remarkably lower in 2 out of 4 horses (25% and 30%) compared to the test conditions. A potential explanation of the lower labeling efficiency could be the omission of shaking the reaction mixture every 5 minutes during the incubation period and the addition of a higher amount of SnCl₂. Furthermore, labeling optimization was performed in laboratory conditions with the ^{99m}Tc generator by the hand, while for the clinical study the ^{99m}Tc was transported in a syringe from abroad. Therefore, the period between elution of the ^{99m}Tc generator and start of the labeling process was much higher (>3 h) in the clinical study. This could have led to reduced radiochemical purity, and consequently lowered labeling efficiencies during the clinical study. However, these labeling efficiencies are comparable with previously reported studies. Considering the post-labeling viability of the ePB-MSCs, high cell-viability was found for 3 out of 4 horses. However, the low post-labeling viability for one horse (31%) combined with the low labeling efficiency did not generate enough counts during the scintigraphy examination. Therefore, the results of horse 3 were excluded from the analysis.

In order to confirm the in vivo stability of the ^{99m}Tc labelled ePB-MSCs complex during the scanning period, the biodistribution of free ^{99m}Tc in horses was evaluated. All four horses were successfully injected with free ^{99m}Tc (min-max: (7.26-7.53 GBq)). On the 1 h and 6 h post-injection scans, accumulation was predominantly observed in stomach, thyroid, bladder and parotid glands. This distribution pattern is comparable with other species such as humans, dogs, cats and rodents. As ^{99m}Tc is a single charged ion with a comparable size to the iodine ion; it is easily bound to plasma proteins and rapidly distributed to the external fluids, leading to the gastric mucosa of the stomach, salivary glands and thyroid gland. For the parotid glands and stomach, a decrease in radioactivity was seen 6 h post-injection in 3 out of 4 horses. Concerning the thyroid gland, no conclusion could be drawn regarding biodistribution in function of time. Furthermore, the known partial excretion route of ^{99m}Tc via the glomerular filtration explains the high radioactivity in the bladder, which clearly decreased in time. A smaller amount of radioactivity was seen in the lungs at both time points, and slowly decreased after 6 h. Finally, an indistinct amount of radioactivity was seen in the intestines, but was not possible to quantify.

The ^{99m}Tc labelled ePB-MSCs accumulated primarily in the lungs. Furthermore, a smaller but considerable amount of radioactivity was seen in the kidneys 1 h post-injection and further increased for all horses after 6 h. The accumulation of ePB-MSCs in both organs increased after the 6 h scan and could be explained by trapping of the cells in the microvasculature of the lungs and kidneys. The presence in the lungs remained constant over time (6 h), which indicates a stable residence in this tissue. However, a transient vascular lung trapping has been described in rats and showed a redistribution from the lungs to other organs at later time points (20 h post-injection). Nevertheless, the presence of ePB-MSCS in the lung and kidney vasculature did not give rise to any adverse effects, which is very important towards safety of IV injected MSCs. A small portion of radioactivity was seen in the bladder, however, as they cannot pass the glomerular filtration, this could not be coming from the ePB-MSCs. This is potentially caused by a limited dissociation of the ^{99m}Tc label from the cells in the kidneys, which is directly secreted in the bladder. Finally, negligible radioactive accumulation was found in the other organs of interest (parotid glands and thyroid) on the scans with ^{99m}Tc labelled ePB-MSCs.

As described above, free ^{99m}Tc is preferentially taken up by the thyroid gland, parotid gland and stomach. As there was no radioactive signal seen in neither of these organs at 1 h and 6 h post-injection of ^{99m}Tc labelled ePB-MSCs, we can conclude the ^{99m}Tc labelling technique gave rise to a stable in vivo complex with ePB-MSCs.

Furthermore, no significant effect on the ePB-MSCs viability after IV injection, as dead cells would also cause accumulation of free ^{99m}Tc in above mentioned organs.

The present invention is in no way limited to the embodiments described in the examples. On the contrary, methods according to the present invention may be realized in many different ways without departing from the scope of the invention.

1. A cell composition comprising isolated mesenchymal stem cells (MSCs), wherein said MSCs are radiolabeled with technetium-99m (^{99m}Tc), characterized in that said radiolabel is essentially free of hexamethylpropylene amine oxime (HMPAO), and wherein said radiolabeled MSCs have a post-labeling cell viability of at least 55%.

2. Cell composition according to claim 1, characterized in that said technetium-99m is reduced by means of stannous chloride towards technetium-99m stannous chloride (^{99m}Tc-SnCl₂).

3. Cell composition according to claim 1, characterized in that the post-labeling cell viability of said radiolabeled MSCs is at least 60%.

4. Cell composition according to claim 1, characterized in that said cell composition comprises at least 60% radiolabeled MSCs, preferably between 65 and 95%, preferably between 75 and 95%, preferably between 85 and 95% labeled MSCs.

5. Cell composition according to claim 1, characterized in that said MSCs are from mammalian origin, preferably of human, equine, canine, or feline origin, more preferably equine origin.

6. Cell composition according to claim 1, characterized in that said cell composition further comprises one or more pharmaceutical active ingredients and wherein said composition is formulation for administering to a patient, preferably intravenous, intraarticular, subcutaneous, intramuscular, or intralesional administration.

7. Cell composition according to claim 1, characterized in that said cell composition further comprises unlabeled isolated MSCs, and/or one or more pharmaceutical agents.

8. Cell composition according to claim 1, for use in biodistribution.

9. Cell composition according to claim 1, for use in diagnostics.

10. Cell composition according to claim 1, for use in cell-based therapies.

11. Cell composition according to claim 1, for use in the prevention or treatment of liver injuries and/or diseases.

12. Cell composition for the use according to claim 11, characterized in that said cell composition is administered to a patient, preferably intravenous, intraarticular, subcutaneous, intramuscular, or intralesional administration.

13. A method for preparing radiolabeled MSCs, characterized in that the method comprises the step of incubating a dose of ^{99m}Tc and a source of post-transition metal ions, preferably stannous ions, with MSCs in the presence of a buffering composition, wherein said buffering composition has a pH of between 7.5 and 9.5.

14. Method according to claim 13, characterized in that the step of incubating lasts at most 1 hour at room temperature, preferably 15 to 40 minutes at room temperature.

15. Method according to claim 13, characterized in that said source of post-transition metal ions is a stannous chloride solution and the buffering composition has a pH of between 8 and 9.

16. Method according to claim 13, characterized in that the dose of ^{99m}Tc is between 10 and 150 millicurie (mCi), preferably between 25 and 96 mCi.

17. Method according to claim 13, characterized in that the method further comprises the step of removing at least an amount of unbound radiolabel.
